(19) United States
(12) Patent Application Publication
(10) Pub. No.: US 2023/0136115 A1
(43) Pub. Date: May 4, 2023

(54) METHOD FOR EVALUATING NEURONAL FUNCTIONAL CONNECTIONS

(71) Applicants: Shusaku SHIOMOTO, Kanagawa (JP); Waka Lin, Tokyo (JP); Koichi Muramatsu, Kanagawa (JP)

(72) Inventors: Shusaku SHIOMOTO, Kanagawa (JP); Waka Lin, Tokyo (JP); Koichi Muramatsu, Kanagawa (JP)

(73) Assignee: Ricoh Company. Ltd., Tokyo (JP)

(21) Appl. No.: 18/051,559

(22) Filed: Nov. 1, 2022

(30) Foreign Application Priority Data
Nov. 2, 2021 (JP) 2021-179391

(51) Int. Cl. G01N 15/14; G01N 33/50; C12N 5/0793

(52) U.S. Cl. CPC G01N 15/1456; G01N 33/5005; C12N 5/0619; G01N 2001/2873

(57) ABSTRACT

A method for evaluating neuronal functional connectivity can be used as a model for evaluating functional repair against damage to neuronal populations. The method can be used for multiple cell populations that are spaced apart from each other and connected via neurites, A method for evaluating neuronal functional connectivity in vitro involves cutting the neurites; observing the dynamics of neurons whose neurites have been cut; and measuring the electrical activity of each of the plurality of cell populations before and after the cutting. At least one of the cell populations contains neurons.

### BACKGROUND OF THE INVENTION

### Field of the Invention

The present invention relates to a method for evaluating functional connectivity of neurons in vitro.

### Description of Related Art

The brain is a biological tissue with a structure in which multiple types of neurons collectively form regions and interact in a complex manner. The brain functions when excitatory and inhibitory neurons are arranged in an appropriate balance in each region of the brain and a circuit network is formed that enables information transmission through synapses, which are the connecting structures between them.

Here, it is generally known that the damaged central nervous system (brain and spinal cord) does not regenerate. It is believed that this is due to the peculiarities of neurons (the inability to divide and the inability of adult neurons to regenerate axons). Much attention has been paid to the regeneration of the central nervous system, since damage to the central nervous system has a particularly large impact on a patient's QOL.

Animal and human cells cultured in vitro are widely used to predict phenomena that occur in vivo. In particular, since iPS cell-derived differentiated cells have become widespread, human neurons, which were previously difficult to obtain, can now be the subject of research, and detailed studies of neurons can be undertaken.

Spatial control of cells is a method for imparting functions to cell tissues in in vitro models. By applying microfabrication technology such as photolithography, it has become possible to freely arrange cell bodies and neurites, which are the components of neurons, and the creation of more complex cell tissue models has also become possible.

Patent Document 1 discloses a method for evaluating cells in vitro, wherein in a plurality of cell populations spaced apart from each other and connected to each other via neurites, the electrical activity of at least two cell populations is measured, wherein at least one of the at least two cell populations whose electrical activity is measured is a cell population comprising at least one type of neuron, and the at least two cell populations whose electrical activity is measured Methods for evaluating cells in vitro, cell substrates, and methods of preparing cell substrates that exhibit different electrical activity characteristics at certain time points are described. This technique seeks to provide an in vitro cell evaluation method that can evaluate electrophysiological interactions between cell populations with different electrical activity characteristics.

Non-Patent Document 1 discloses a lab-on-a-chip platform that enables cutting of axons and arrangement of neurons as a multi-node network.

However, in the conventional in vitro evaluation system involved in neuronal regeneration, there was a lack of an evaluation model for functional connections between neuronal populations. For example, although there have been methods for artificially reconnecting from the outside after functional connections between neuronal populations have been destroyed in vitro, there has been insufficient research on spontaneous repair (functional reconnection). Patent Document 1 by the present inventors reports results of spontaneous repair after disruption of functional connections between cell populations including neurons, and action potential data. In Patent Document 1, isolated cell populations are electrically connected by being connected by neurites, and the electrical connection is cut off by cutting the neurites. In addition, it has been electrically evaluated that the connection is restored by repairing. However, in Patent Document 1 and Non-Patent Document 1, data indicating the process of neurite repair of the neuronal population could not be obtained.

In addition, although research on spinal cord injury has been conducted in rodents, electrical functional connectivity at the cellular level has not been evaluated. In other words, the current situation is that the behavior (locomotion), which is a more advanced evaluation, is being evaluated instead of the cellular level. In addition to behavioral evaluation, it is desirable to understand cellular level repair mechanisms. Understanding these will enable the selection of drug candidates that promote regeneration of nerve function, so it is desirable to obtain these findings.

Also, in the above rodent research, it was difficult to evaluate functional connectivity in the case of cranial nerves. In addition, animal experiments have problems such as species differences, animal welfare, and the inability to conduct large- scale experiments.

### SUMMARY OF THE INVENTION

An object of the present invention is to provide a model for evaluating functional repair of damaged neuronal populations.

A method for evaluating neuronal functional connectivity according to the present invention is a method for evaluating functional connectivity of neurons in vitro, comprising: a cutting step of culling the neurite; an observation step of observing the dynamics of the neuron with the cut neurite; and an activity measurement step, wherein at least one of the cell populations includes neurons.

The present invention can provide a model for evaluating functional repair of damaged neuronal populations.

### BRIEF DESCRIPTION OF THE DRAWING

FIG. 1 is a plan view showing a cell substrate according to the first embodiment of the present invention.

FIG. 2 is a cross-sectional view showing the cell substrate cut along line II-II shown in FIG. 1.

FIG. 3 is a plan view showing a cell substrate according to a second embodiment of the present invention.

FIG. 4 is a diagram showing a test flow in Example 1.

FIG. 5A is a photographic diagram and a schematic diagram showing the arrangement of cells in Example 1.

FIG. 6 is a diagram showing measurement data of electrical activity in Example

FIG. 7 is a schematic diagram showing a jig used in Example 2, and a diagram showing a photograph of cell arrangement and image observation.

FIG. 8 is a diagram showing a photograph of image observation in Example 2.

FIG. 9 is another diagram showing a photograph of image observation in Example 2.

FIG. 10 is diagrams showing analysis results by image observation in Example 2.

### DETAILED DESCRIPTION OF THE INVENTION

### Method for Evaluating Functional Connections of Neurons

In an embodiment, the present invention provides a method for assessing functional connectivity of neurons in vitro, wherein a plurality of cell populations spaced apart from each other and connected via neurites are subjected to a cutting step of cutting the neurites; an observation step of observing the dynamics of the neurons whose neurites have been cut; and an electrical activity-measuring step of measuring the electrical activity of each of the plurality of cell populations before and after the cutting step; wherein at least one of said cell populations comprises a neuron.

Neuronal functional connection refers to the fact that functional actions are produced by physically connecting neurons (nerve cell) to each other or to other cells. Connections broadly include physical connections, but in particular, refer to connections between neurites extending from neurons and other cells. Functional connection means that the above-mentioned physical connection causes interaction in terms of, for example, electrical and chemical functions, and through these actions, functions of a plurality of cells are produced. In particular, it refers to the occurrence of an electrical action.

Evaluation methods broadly refer to methods of obtaining and analyzing information on the physical and functional states of cells. Preferably, they refer to composite evaluation and analysis of the plurality of states.

As will be described later in Examples, in this embodiment, by the process of functional restoration (observation of neurite dynamics) and the result of functional restoration (cell population by combining these evaluations), it is possible to obtain an evaluation of the relationship between kinetics and functional connections in cell repair. Therefore, the present embodiment can provide a model for evaluating functional repair of damaged neuronal populations.

### Cell Population

As used herein, the term “cell population” refers to a collection of cells containing two or more cells, and may be a cell mass configured by adhering cells to each other. Moreover, it may be formed by a single type of cells, or may contain multiple types of cells. Cell populations also exhibit specific electrical activity characteristics as a population.

The cell populations are spaced apart from each other and connected via neurites.

The distance between cell populations is not particularly limited as long as connection via neurites is achieved, but is preferably 100 μm or more. When the separation distance is equal to or greater than the lower limit value, the action potentials of the cell populations are not mixed up and can be measured with higher accuracy. On the other hand, the upper limit of the distance can be about 3 cm, for example, considering the distance that the neurites can extend. Typically, about 100 μm or more and 1 cm or less is preferable.

Moreover, when arranging the cell populations, as in the examples described later, the cell populations are arranged in separate culture spaces, and the culture spaces are separated from each other by micrometers on the order of μm (at least one piece is less than 1000 μm in size). It is also preferable that they be connected via a tunnel. Cell bodies cannot enter microtunnels, but cell projections can, allowing cell projections to be controlled in more detail, and enabling more detailed examination of the neurite dynamics of cells.

At least one of the cell populations whose electrical activity is measured contains neurons. Neurons can be broadly classified into, for example, peripheral nerves and central nerves, Peripheral nerves include, for example, sensory neurons, motor neurons, and autonomic neurons. Central nerves include, for example, interneurons and projection neurons. Projection neurons include, for example, cortical neurons, hippocampal neurons, amygdala neurons, and the like. Central neurons can also be broadly classified into excitatory neurons and inhibitory neurons. Examples include glutamatergic neurons mainly responsible for excitatory transmission in the central nervous system, GABA (γ-aminobutyric acid) agonistic neurons mainly responsible for inhibitory transmission, and the like. Other neurons that release neuromodulators include cholinergic neurons, dopaminergic neurons, noradrenergic neurons, serotonergic neurons, histaminergic neurons, and the like.

Other cell populations other than at least one cell population containing neurons are connected to at least one cell population containing neurons via neurites, and transmit signals from the at least one cell population. Receivable cells are included. Cells capable of receiving such transmission signals include, for example, neurons, muscle cells, and the like. Muscle cells include, for example, cardiomyocytes, skeletal muscle cells, smooth muscle cells, and the like. These cells may be used singly or in combination of two or more. Cells capable of receiving transmission signals contained in the other cell population are preferably neurons from the viewpoint that a network can be constructed in the entire evaluation system. That is, since all cell populations can exchange signals with each other via neurites, it is preferable that all cell populations in the evaluation system contain neurons.

Cells contained in these cell populations may be primary cultured cells, subcultured cells, established cells, immortalized cells, and various others. They may be gene-edited cells. In addition, cells induced to differentiate from stem cells are preferable from the viewpoint that a cell population containing many desired cells can be easily obtained. Stem cells include, for example, embryonic stem cells, induced pluripotent stem cells, mesenchymal stem cells, cord blood-derived stem cells, neural stem cells and the like. Examples of induced pluripotent stem cells include nuclear transplanted embryonic stem cells (ntES cells), induced pluripotent stem cells (iPS cells), and the like. Examples of mesenchymal stem cells include bone marrow mesenchymal stem cells and adipose tissue-derived mesenchymal stem cells. Among them, iPS cells are preferable as stem cells. The iPS cells may be derived from healthy subjects or from patients with various nervous system diseases. Moreover, various gene-edited cells may be used, for example, gene-edited cells may have genes that are causative agents or risk factors for various nervous system diseases. When iPS cells are cells derived from patients with various nervous system diseases, they can be used to construct disease models of the nervous system. Neurological disorders include, but are not limited to, neurodegenerative disorders, autism, epilepsy, attention-deficit hyperactivity disorder (ADHD), schizophrenia, bipolar disorder, and the like. Examples of neurodegenerative diseases include Alzheimer's disease, Parkinson's disease, amyotrophic lateral sclerosis and the like.

Animal species from which cells are derived are not limited, but include, for example, humans, monkeys, dogs, cows, horses, sheep, pigs, rabbits, mice, rats, guinea pigs, and hamsters. Among them, mammals are preferable, and humans are particularly preferable.

The cells contained in the cell population may be those collected from the living body as described above, those that have been established and cultured, or those that have been induced to differentiate from stem cells. As the cell population, the cells obtained as described above may be used as they are, or may be formed by mixing other cells. The cell population is preferably stem cell-derived, that is, a cell population obtained by inducing differentiation of stem cells, from the viewpoint of easily obtaining a cell population having desired characteristics. When used in the evaluation method of the present embodiment, all cell populations are preferably derived from stem cells from the viewpoint of facilitating construction of a desired evaluation system.

When multiple cell populations are connected via neurites, it specifically means that they are functionally connected. Functional connection refers to a state in which cells can exchange electrical signals with each other via neurites.

### Cutting Step

In one aspect of the present invention, the cutting step comprises cutting at least one of the neurites connecting the cell population.

In the cutting step, some of the neurites that connect the cell populations may be cut. By cutting some of the cell population, it is possible to obtain data on behavior such as repair when some of the connections of the cell population are cut and some remain, according to this evaluation method of functional connection of neurons. Moreover, in the cutting step, all of the neurites that connect the cell populations may be cut. By cutting all of them, the present evaluation method for neural cell functional connections makes it possible to obtain data on behavior such as repair when all connections of a cell population are severed. By appropriately selecting and comparing these data, it is possible to obtain data on nerve tissue damage and its repair.

For the cutting step, any means capable of cutting the neurites can be used as appropriate. For example, mechanical cutting means, thermal cutting means, or the like can be used. As the mechanical cutting means, for example, cutting with a scalpel or a needle can be used. These cutting means may use an automatic control device such as a manipulator to control cutting.

### Observation Step

This embodiment includes, after the cutting step, an observation step of observing the dynamics of neurons whose neurites have been cut.

For the observation step, any means that can observe the dynamics of neurons may be used as appropriate. For example, image observation, observation with an optical sensor or an electric sensor, observation of transmittance, optical imaging, observation of electrical behavior, and the like may be performed. Images may be observed through various staining means, wavelengths, and the like. A sensor or the like that converts light into an electric signal may be used, and examples of such a sensor include a CMOS sensor and the like. In this embodiment, in particular, it is possible to observe the dynamics of neurons using a camera.

The observation step may be performed continuously or intermittently. As a continuous process of the step, at least one observation for a period of time after cutting may be performed. The continuous process may also include at least one continuous measurement process before and after cutting. As an intermittent process, short measurements may be taken at least once after cutting.

For example, when performing intermittent observations multiple times, observation is performed once immediately after the cutting step, and then observation is performed multiple times after a certain period of time, so that the process of repairing the cut neurites can be observed. The observation period and number of times depends on the cell repair time and culture time.

In the continuous process of image observation, it is possible to observe, for example, video shooting, time lapse. etc. As an intermittent process, for example, still image photography can be performed.

Also, as the image observation, the electrical behavior in the image may be observed. For example, various means such as electrical measurement and electrode measurement can be used. Further, as a specific example, a means for tracing the axonal region by a sensor placed under the cell can be considered. As such means, a tunnel CMOS electrode or the like can be used.

### Electrical Activity Measurement Step

This embodiment includes an electrical activity measurement step of measuring the electrical activity of each of the plurality of cell populations before and after the cutting step. Measuring electrical activity broadly includes the steps of detecting, measuring, and analyzing electrical activity characteristics, as described later, and preferably includes measuring temporal changes in the electrical activity characteristics associated with cell activity.

“Before and after the cutting step” means that the electrical activity measurement step is performed before and after the cutting. In particular, it is preferable to carry out at least one measurement each before and after said cutting step. The electrical activity measurement step performed before and after the cutting step may be performed continuously or intermittently. As a continuous process, there may be at least one measurement at a fixed time before cutting and one measurement at a fixed time after cutting. The continuous process may also include at least one continuous measurement step before and after cutting. As an intermittent process, short-term measurements may be taken at least once before and after each cutting.

As used herein, the term “electrical activity characteristics” refers to properties related to electrochemical activity exhibited by cell activity, such as the firing frequency, firing amplitude, firing pattern, synchronous burst of a cell population, burst frequency, burst amplitude, burst pattern, burst periodicity, oscillation frequency of nerve oscillation, oscillation amplitude, oscillation phase, intracellular cation concentrations such as sodium ions and calcium ions, and their change patterns. Here, cell firing may be spontaneous or stimulated, and includes action potential. it is possible to observe only a single electrical activity characteristic, but in recent years, multiple characteristics (parameters) have been simultaneously measured and characterized using multivariate analysis. A combination of these characteristics is also included in the “electrical activity characteristics” of the present invention. Electrochemical properties that can be measured using electrodes are preferable in terms of ease of measurement, and the firing frequency, firing amplitude, and firing pattern of cell firing, the burst frequency, burst amplitude, and burst pattern of synchronous bursts of cell populations. For the measurement, it is preferable to use cell populations in which at least one selected from the group consisting of burst periodicity, oscillation frequency of nerve oscillation, oscillation amplitude and oscillation phase is different. Electrical activity properties of a cell or cell population can be measured using known electrophysiological techniques. Examples of such electrophysiological techniques include, but are not limited to, a method of measuring local electric field potentials using electrodes such as a multi-electrode array, a method of directly measuring action potentials by a patch clamp method and the like, a method of measuring changes in membrane potential using a membrane potential-sensitive dye or the like, a method of measuring cation fluctuations such as a calcium imaging method, and the like. Membrane potential-sensitive dyes include, for example, calcium-sensitive dyes composed of a calcium chelator and a fluorophore, styryl-based compounds, cyanine- and oxonol-based compounds, rhodamine derivatives, and the like.

The evaluation method of the present invention includes measuring the electrical activity of cell populations as described above. Electrical activity measurements are performed on at least two cell populations, and may be performed on, for example, two, three, four, five, or more cell populations. These cell populations are spaced apart from each other in plan view. At least two cell populations whose electrical activity is measured are connected to each other via neurites (axons). As used herein, “connected to each other via neurites” means that signals are transmitted from one cell population to another cell population via neurites, typically a synchronous ignition or burst is observed. When there are three or more cell populations whose electrical activity is measured, connection via neurites is not particularly limited as long as a signal generated in a certain cell population is transmitted to all cell populations. It may be concatenation or parallel concatenation. For example, each cell population may be independently connected to each other in parallel, all cell populations may be connected in series, or they may be mixed.

As used herein, “measuring electrical activity” means measuring changes over time in the electrical activity characteristics associated with cell activity. By measuring the electrical activity of at least two or more cell populations connected to each other via neurites, it is possible to understand how these cell populations exchange signals, and in turn, how each cell population is organized. It is possible to evaluate how they interact with each other. Measurement of electrical activity may be performed as long as the electrical activity can be evaluated, and may be performed multiple times. In one aspect of the invention, electrical activity is measured continuously during the test.

The at least two cell populations whose electrical activity is measured may or may not have the same electrical activity characteristics at the time the electrical activity is measured. When the electrical activity characteristics are different, the cell populations originally having different electrical activity characteristics may be used. If they have different electrical activity characteristics, this may be as a result of changes in electrical activity characteristics due to the addition of, for example, NMDA receptor inhibitors, etc. or communication from another cell population via neurites. Such changes in electrical activity characteristics should be achieved by the time the electrical activities of at least two cell populations are measured.

As mentioned above, in the past, it was only possible to observe synchronous bursts due to random neural network connections within a single cell population, and it was not possible to accurately evaluate drugs that act on neurons. In other words, in actual brain tissue, various neural circuits are intricately constructed, such as feedback in which changes in electrical activity downstream of the signal act on the upstream of the signal, so electrical activity in a single cell population can be simply confirmed. However, it was not possible to accurately evaluate the cells and the drugs that act on them. According to the evaluation method of the present embodiment, the influence of a plurality of different cell populations, such as synchronous effects and feedback effects, can also be evaluated, so cells and drugs that act on them can be evaluated more accurately than before.

In one aspect of the present invention, electrical activity is measured using electrodes. It is preferable to use electrodes because electrical activity can be directly and easily measured. As such electrodes, for example, in addition to electrodes capable of measuring local electric field potentials such as multi-point planar electrodes (MEA), electrodes capable of directly measuring cell action potentials such as micro glass electrodes also can be used. Aspects using such electrodes will be described in detail in the section on cell substrates along with specific examples.

The evaluation method of this embodiment can evaluate the properties of each cell population and the correlation between cell populations by confirming the firing and the like in the state in which the evaluation system is constructed. For example, when observing the firing of arbitrary cell populations A and B connected via neurites, if firing synchronized with the firing of cell population B can be confirmed in addition to the firing of cell population A, the cell population A can be evaluated as receiving a transmission signal from the cell population B. In this case, if cell population B cannot be confirmed to be firing in synchronization with the firing of cell population A, it can be evaluated that cell population B is upstream of cell population A and is not receiving feedback projection.

A preferred embodiment of the evaluation method of the present invention includes adding a drug presumed to act on neurons (referred to as a “candidate drug” in the present disclosure) to a cell population whose electrical activity is to be measured. If the addition of the candidate drug results in any change in electrical activity, it can he determined that the candidate drug acts on neurons. This makes it possible to efficiently select candidate drugs that act on neurons. Thus, in one aspect of the invention, the method comprises evaluating the effect of a candidate agent on cell populations (or neurons residing therein), and in a further aspect, screening for effective candidate agents based on such evaluation is included. That is, the present invention includes a screening method for drug candidates using the evaluation method of the present invention. The details of the evaluation method of the present invention will be described in detail in the section on cell substrates together with specific examples.

### Function and Effect of the Embodiment

In the measurement method of this embodiment, by observing the dynamics of neurons after cutting cells, it is possible to obtain data on the dynamics of neurons, especially the process of repairing neurites after cutting.

In addition, by analyzing cell dynamics and measuring electrical activity together, it is possible to obtain data on the correlation between neuronal dynamics and function, especially the correlation between the repair of neuron processes and the recovery of electrical activity functions.

By measuring the change in electrical activity in the cell population whose electrical activity was measured other cutting the cell projections in the cutting step, it is possible to evaluate how the communication through such neurites affected the population. In addition, by confirming the effect of the candidate drug in an evaluation system in which the neurites are cut, the candidate drug can be evaluated as a model fix traumatic brain injury or the like.

### Cell Substrate

Next, a cell substrate that can be suitably used in the method for evaluating neuronal functional connections according to this embodiment will be described. The cell substrate of the present invention can be used particularly for evaluating cells in vitro. The cell substrate of the present invention will be described in detail below using specific examples of embodiments. Further, the details of the evaluation method of this embodiment will be specifically described in the method of using the system for evaluating cells in vitro shown below.

### First Embodiment

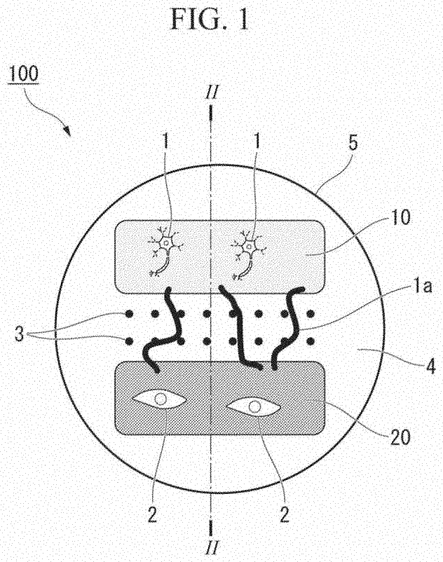

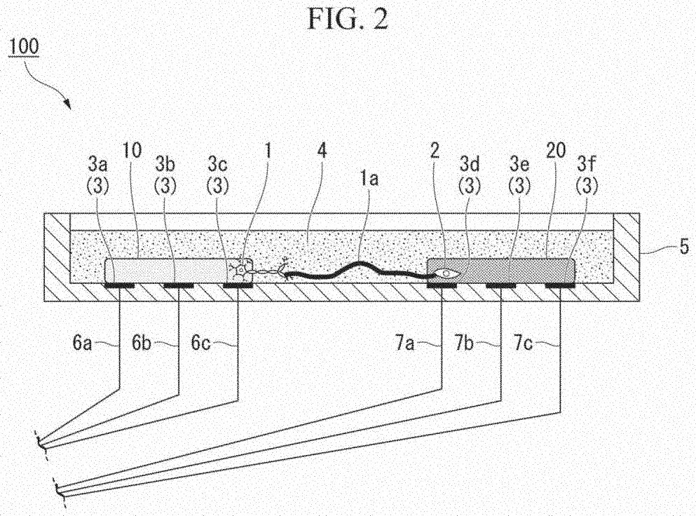

FIGS. 1 and 2 are diagrams showing a cell substrate according to the first embodiment of the present invention. FIG. 1 is a plan view showing a cell substrate 100 according to the first embodiment of the invention. FIG. 2 is a cross-sectional view of the cell substrate 100 cut along line II-II shown in FIGS.

The cell substrate 100 includes a substrate 5 having a plurality of detection units 3, and a first cell population 10 and a second cell population 20 arranged separately from each other in plan view on different detection units 3. A medium 4 is filled on the substrate 5. The first cell population 10 and the second cell population 20 exhibit different electrical activity characteristics and form connections via neurites 1a. In the evaluation method of this embodiment, by using the cell substrate having the above configuration, electrophysiological interactions between cell populations having different or the same electrical activity characteristics can be evaluated.

As shown in FIG. 2, the first cell population 10 is arranged on detection units 3a, 3b and 3c embedded in the substrate 5. The first cell population 10 may be arranged on one or more detection units 3, but is preferably arranged on two or more detection units 3. As a result, the electric field potential generated by the action potential change of a plurality of cells contained in the first cell population 10 can be detected in each detection unit 3, and the average value of the obtained electric field potentials is calculated, and highly accurate electric field potential data can be obtained.

### First Cell Population

In the present embodiment, at least one of the first or second cell population contains neurons, but in the illustrated example, the first cell population 10 is a neuron population containing one or more neurons 1. The first cell population 10 may be a cell population consisting only of neurons 1 as long as the population exhibits specific electrical activity characteristics, or may be a mix of cell of neurons 1 and cells other than the neurons 1. Further, when the first cell population 10 is a cell population consisting only of neurons 1, it may be a cell population consisting of a group of one kind of neurons 1, or a cell consisting of mixed cells of two or more kinds of neurons 1.

The content of neurons in the neuron population is not particularly limited, but is usually 1% or more, preferably 5% or more, more preferably 20% or more, further more preferably 40% or more, and particularly preferably 80% or more, for the total number of neurons.

As the neuron 1, the neuron described above can be used.

Cells other than the neuron 1 included in the first cell population 10 include, for example, glial cells. Glial cells are roughly classified into astrocytes, oligodendrocytes, microglia, ependymal cells, Schwann cells and the like.

As the animal species from which the cells contained in the first cell population 10 are derived, the animal species described above can be used.

### Second Cell Population

The second cell population 20 is a cell population containing one or more cells 2 (hereinafter sometimes simply referred to as “cells 2”) that transmit electrical signals to the neurons 1.

The second cell population 20 may be a cell population consisting only of the cells 2, as long as it exhibits a synchronous action potential in response to an external stimulus, the cells 2 and cells other than the cells 2, and it may be a cell population consisting of mixed cells. Further, when the second cell population 20 is a cell population consisting only of cells 2, it may be a cell population consisting of one type of cells 2, or a cell population consisting of mixed cells of two or more types of cells 2.

The content of cells 2 in the second cell population is not particularly limited, but is usually 1% or more, preferably 5% or more, more preferably 20% or more, and further preferably 40% or more, of the total number of cells in neurons.

Cells 2 include, for example, neurons, muscle cells, and the like. Muscle cells include, for example, cardiomyocytes, skeletal muscle cells, smooth muscle cells, and the like. These cells may be used singly or in combination of two or more.

Examples of cells other than the cells 2 contained in the second cell population 20 include, in addition to the above glial cells, cells other than muscle cells contained in heart and muscle tissue such as fibroblasts and vascular endothelial cells.

Cells contained in the second cell population 20 include those similar to those exemplified for the first cell population 10 above.

The animal species from which the cells contained in the second cell population 20 are derived include those exemplified for the first cell population 10 above.

### Culture Medium

The medium 4 is a basal medium containing components necessary for survival and growth of cells (inorganic salts, carbohydrates, hormones, essential amino acids, non- essential amino acids, vitamins), contained in the first cell population 10 and the second cell population 20. The medium 4 can be selected as appropriate depending on the type of cell. For example, a basal medium with necessary components added, such as Dulbecco's Modified Eagle's Medium (DMEM), Minimum Essential Medium (MEM), RPM1-1640, Basal Medium Eagle (BME), Dulbecco's Modified Eagles Medium: Nutrient Mixture F-12 (DMEM/F-12), Glasgow Minimum Essential Medium (Glasgow MEM) is included, but is not limited thereto. Commercially available media may be used as media for culturing neurons, and examples of such media include BrainPhys (Stemcell Technologies), Neurobasal, Neurobasal Plus (both Thermo Fisher Scientific) and the like.

### Detection Unit

The detection unit 3 is configured to detect electric field potentials generated by action potential changes in each cell contained in the first cell population 10 and the second cell population 20. It should be noted that “detecting” here includes detecting the generation of electric field potential and detecting specific frequency, amplitude and phase changes in electric field potential. The detection unit 3 may be embedded in the substrate 5 so that the surface of the detection unit 3 is exposed and in contact with each cell population, or may be arranged on the substrate 5.

The number of detection units 3 may be 2 or more, for example, 4, 8, 16, 32, 64, and the like.

A specific example of the detection unit 3 is an electrode. Since the detection unit 3 is an electrode, it is possible to detect changes over time in the frequency, amplitude and phase of the electric field potential generated by the cell population.

### Substrate

The substrate 5 has the detection unit 3. A specific example of the substrate 5 having the detection unit 3 (especially electrodes) is a multi-electrode array (MEA). in addition, the substrate 5 is preferably configured to hold the medium 4 in order to maintain and culture the first cell population 10 and the second cell population 20. For example, a culture container with wells filled with MEA can be used. The shape of the culture container may be a dish type having one well, or a multiwell plate type having a plurality of wells.

The substrate 5 may be made of any material as long as it is not toxic to cells, but elastic materials, glass, ceramics, metal materials such as stainless steel, etc. are preferable. Examples of elastic materials include cycloolefin, polystyrene, polyethylene, polypropylene, polycarbonate, polyamide, polyacetal, polyester (polyethylene terephthalate, etc), polyurethane, polysulfone, polyacrylate, polymethacrylate (polymethyl methacrylate (PMMA), etc.), polyvinyl, etc. silicone resins such as PDMS (poly-dimethylsiloxane); synthetic rubbers such as EPDM (ethylene propylene diene monomer); and natural rubbers.

For the substrate 5, these materials can be used singly or in combination of two or more.

### Second Embodiment

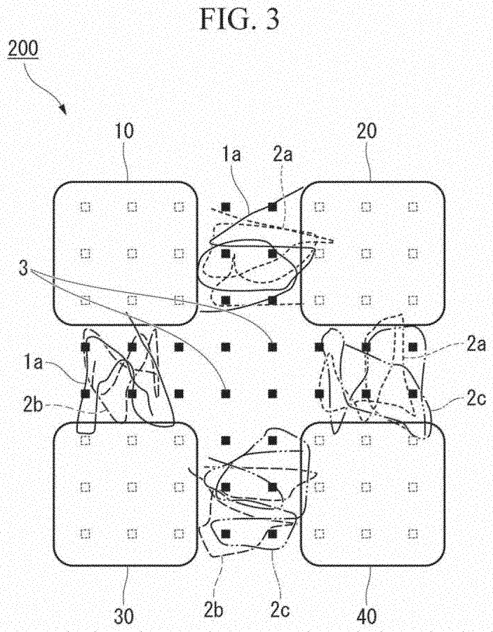

FIG. 3 is a plan view showing a cell substrate 200 according to the second embodiment of the invention. The cell substrate 200 shown in FIG. 3 is different from the cell substrate 100 shown in FIGS. 1 and 2 in that it has four cell populations including a third cell population and a fourth cell population in addition to the first cell population and the second cell population. By providing four cell populations in the cell substrate 200, more complex neurite-mediated connections can be constructed, and electrophysiological interactions between the four cell populations can be evaluated.

### Third Cell Population and Fourth Cell Population

The third cell population 30 and the fourth cell population 40 are placed on the detection unit 3 other than the detection unit 3 where the first cell population 10 and the second cell population 20 are arranged, in plan view. They can be arranged apart from the cell population 20.

The third cell population 30 and the fourth cell population 40 are cell populations that exhibit synchronous action potentials in response to external stimuli. The third cell population 30 and the fourth cell population 40 may be cell populations composed of the same cells as the first cell population 10 or the second cell population 20, or cell populations consisting of different cells may be used. The third cell population 30 and the fourth cell population 40 can form a connection via neurites with at least one of the first cell population 10 and the second cell population 20. As shown in FIG. 3, the first cell population 10 and the third cell population 30 form connections via neurites 1a and 2b, and the second cell population 20 and the fourth cell population 40 form neurites 2a and 2c. The third cell population 30 and the fourth cell population 40 may also form connections via neurites 2b and 2c.

Cells contained in the third cell population 30 and the fourth cell population 40 include those similar to those exemplified in the first cell population 10 and the second cell population 20 above.

The cell substrate is not limited to those shown in FIGS. 1 to 3, and other configurations may be used.

For example, in the cell substrate 100 shown in FIGS. 1 and 2, the second cell population 20 may be a cell population containing one or more neurons of a different type from the neuron 1. Neurons contained in the second cell population 20 include those exemplified for the first cell population 10 above.

For example, in the cell substrate 200 shown in FIG. 3, the number of cell populations is not limited to 4, and may include a plurality of 5 or more cell populations.

### Method for Producing Cell Substrate

A cell substrate can be produced, for example, by the method shown below.

First, a partition member is placed between regions in which a plurality of cell populations are to be placed on a substrate having a detection portion (electrodes, etc.). At this time, in order to keep each cell population within a certain area on the substrate, a frame member may be arranged so as to surround each cell population. The partition member and the frame member may be made of any material as long as it is not toxic to cells, and examples thereof are the same as those exemplified as the material of the substrate 5. Moreover, as a cell population, the cell population detailed above can be used.

Next, a plurality of cell populations are arranged so as to be separated from each other in plan view with the partition member interposed therebetween. Alternatively, when using a substrate on which a frame member is further arranged, each cell population is arranged so as to fit within the region surrounded by the frame member.

As a method for arranging the cell population, a cell suspension is prepared by mixing the cell population with a medium or a buffer solution, and the cell suspension is seeded using a micropipette, or the arrangement area is 10 mm² or less. In the case of a narrow area, a method of seeding the cell suspension using an inkjet bioprinter and the like can be used. In particular, when seeding using a high-precision bioprinter, each cell population can be spaced apart at a narrow distance without a partition member or the like, which is preferable.

After arranging each cell population, it is cultured until each cell population adheres to the substrate. As culture conditions, for example, the cells can be cultured under normal cell culture conditions such as 37° C. and 5% CO₂ concentration for a certain period of about 10 hours or more and 48 hours or less. In addition, the medium used for culturing can also be appropriately selected according to the type of cells, and examples thereof include those exemplified for the above cell substrates.

After confirming that the cells contained in each cell population have adhered to the substrate, the partition member (and the frame member) is removed and cultured. As culture conditions, the cells can he cultured for about 5 days or more and 60 days or less, for example, for a certain period of 18 days or more and 28 days or less under normal cell culture conditions such as 37° C. and 5% CO₂ concentration. By culturing for a certain period of time, a bond can be formed between the first cell population 10 and the second cell population 20 via neurites.

When the cells contained in the cell population are iPS cell-derived cells, the iPS cells can be used after differentiating into various cells using a known method. For differentiation induction from iPS cells to various cells, a commercially available differentiation induction kit, for example, a differentiation induction kit manufactured by Elixirgen Scientific (specifically, dopaminergic neurons, cholinergic neurons, glutamatergic neurons mixed culture differentiation induction kit of cells and serotonergic neurons; cholinergic neuron differentiation induction kit; dopaminergic neurons differentiation induction kit; GABAergic neurons differentiation induction kit; skeletal muscle cell differentiation induction kit) and so on, can be used. Alternatively, commercially available iPS cell-derived differentiation-induced neurons may be used.

Various cells induced to differentiate from iPS cells can be confirmed to have been induced to differentiate into desired cells by detecting biomarkers specific to the cell type. For example, mature neurons selected from the group consisting of Dcx, MAP-2, synapsin 1, TuJ1, NSE, Map2a, Gap43, NF, CD24, CDH2/CD325, synaptophysin, and CD56/NCAM. A mature neuron can be identified based on expression of at least one marker that is identified as a neuron.

Neurons can also be identified by their morphology having axons and dendrites and by generating action potentials.

A specific type of neuron includes dopaminergic neurons (at least one marker selected from the group consisting of TH, AaDC, Dat, Otx-2, FoxA2, LMX1A and VMAT2), cholinergic neurons (NGF and at least one marker selected from the group consisting of ChAT). GABAergic neurons (at least one marker selected from the group consisting of GAD67 and vGAT), glutamatergic neurons (vGLUT1), serotonergic neurons, motor Neurons (at least one marker selected from the group consisting of HB9, SMN, ChAT and NKX6), sensory neurons (at least one marker selected from the group consisting of POU4F1 and peripherin), astrocytes (at least one marker selected from the group consisting of GFAP and Tapa1), and phenotypic markers characteristic of oligodendrocytes (at least one marker selected from the group consisting of O1, O4, CNPase, and MBP). It can be identified based on whether it is expressed or not.

Neurons with sensitivity to specific neurotransmitters have receptors and enzymes involved in neurotransmitter biosynthesis, release, and reuptake, and depolarization and repolarization events associated with synaptic transmission can be identified based on having ion channels involved in synaptogenesis and can be confirmed by staining for synaptophysin. Specific neurotransmitter receptivity can be confirmed by detecting receptors for, for example, gamma-aminobutyric acid (GABA), glutamate, dopamine, 3,4-dihydroxyphenylalanine (DOPA), noradrenaline, acetylcholine, and serotonin.

The electrical activity characteristics of the cell population used for the cell substrate can be confirmed by measuring the action potential of the cells contained in the cell population, the synchronous burst exhibited by the cell population, etc. using a known electrophysiological method. Specifically, as shown in Examples to be described later, the first cell population 10 and the second cell population 20 are placed on a substrate provided with MEA, for example, MED64 system (manufactured by SCREEN Holdings Co., Ltd.), Maestro MEA (manufactured by Axion Bio Systems), MEA systems (manufactured by Multichannel Systems), or the like, by measuring the action potential for about 5 minutes.

### Evaluation Method using an Evaluation System

The above cell substrate can also be used as a system for evaluating cells in vitro (hereinafter sometimes simply referred to as an “evaluation system”).

### How to Use

The method of using the evaluation system of this embodiment will be explained below.

For example, when using the cell substrate 100 according to the first embodiment of the present invention, the action potentials of the first cell population 10 and the second cell population 20 are detected by the detection unit 3, and a wiring 6a is detected from the detection unit 3, wirings 6b, 6c, 7a, 7b and 7c to a measurement unit (not shown) for analysis. From the measurement result of the action potential of each cell population, one or more selected from the group consisting of the cycle, amplitude and phase of the action potential of the first cell population 10 and the second cell population 20 are partially or wholly synchronized by confirming whether or not there is an electrophysiological interaction between the first cell population 10 and the second cell population 20, and it is possible to determine whether or not there is an electrophysiological interaction.

For example, as shown in Examples described later, when the firing patterns of some of the action potentials of the first cell population 10 and the second cell population 20 are synchronized, the first cell population 10 and the second cell population 20 can be determined.

Furthermore, by artificially cutting the formed bond using a medical scalpel or the like and measuring the action potential of the first cell population 10 and the second cell population 20 after cutting, the electrical activity of each cell population can also be evaluated.

For example, as shown in Examples described later, the electrical activity characteristics of the first cell population 10 and the second cell population 20 after cutting are different from each other as before the formation of connections via neurites. If so, it can he determined that the synchronization of the firing pattern of some of the action potentials is due to the formation of this bond.

For example, when using the cell substrate 200 according to the second embodiment of the present invention, the action potentials of the first cell population 10, the second cell population 20, the third cell population 30, and the fourth cell population 40 are detected by the detection unit 3, and the action potential data is transmitted from the detection unit 3 to the measurement unit through wiring for analysis. This makes it possible to determine which cell populations are affected or influenced by individual cell populations within a complex cell substrate, or which cell populations are not affected.

Furthermore, in the same manner as in the case of using the cell substrate 100, one or more bonds among the bonds formed are cut, and the action potential of each cell population after cutting is measured to measure the electrical activity of each cell population. At this time, all bonds between cell populations may be cleaved, bonds may be partially cleaved only between desired cell populations, or bonds between cell populations may be cleaved sequentially in a desired order.

In addition, after cutting, an observation step of observing the cells on the cell substrate 100 using a camera (not shown) can also be performed to observe the dynamics of the first cell population 10 and the second cell population 20. Specifically, after the bonds formed by the neurites are cut, the process of neurite re-formation can be observed by observing images with a camera multiple times at given intervals.

In addition, by analyzing the evaluation of the kinetics and the electrical activity together, the correlation between the kinetics and the electrical function can be obtained, for example, data on whether the recovery of the connection of cells and the recovery of the electrical function are correlated.

Since the evaluation method and evaluation system of the present embodiment are useful for axonal guidance, neurodegenerative disorders, neuronal plasticity, neuronal development and cytological disorders such as neuronal learning and memory, repair of damaged neurons, and the like, they can be very useful in subsequent behavioral and molecular basic research.

The evaluation method of the present embodiment can provide an evaluation model for functional connections between neuronal populations of cells, and can particularly contribute to research on spontaneous repair (functional reconnection) of neurons. It is possible to evaluate the recovery of connections by repairing neurons from both the dynamic and electrical aspects of cells. Obtaining knowledge about regeneration of the central nervous system can contribute to improving the QOL of patients suffering from damage to the central nervous system.

In addition, the evaluation method and evaluation system of the present embodiment can also be used to evaluate arbitrary compounds. Specifically, after adding an arbitrary compound to one or more cell populations among a plurality of cell populations, the action potential of each cell population is measured. This makes it possible to evaluate the effects of any compound on each cell population, specifically, the ability of any compound to activate neural networks, the ability to change synchronous firing, and the toxicity of the compound. Also, based on such evaluation, a determination can be made as to whether a compound may be useful in treating a particular disease.

For example, as shown in the Examples below, a first cell population rich in excitatory neurons with neurite-mediated connections and a second cell population rich in inhibitory neurons were exposed to a compound. In some cases, when the action potential firing pattern of the first cell population is completely synchronized with that of the second cell population, it can be determined that the compound has the ability to suppress synchronous firing of neurons. In addition, based on the determination, it can be determined that the compound may be useful for treating diseases in which synchronous firing is excessive (e.g., epilepsy, autism, schizophrenia, etc.). Thus, the evaluation method and evaluation system of the present embodiment can be suitably used for compound screening.

### EXAMPLES

Next, the present invention will be described in more detail with reference to examples, but the present invention is not limited to the following examples.

### Example 1

### Construction of an In Vitro Evaluation System Using a Cell Substrate Containing Two Neural Populations

### Preparation of Cells

Two types of frozen iPS cell-derived neurons (GABAergic Neurons from Healthy Donor and Mixed Neurons from Healthy Donor) commercially available from Elixirgen Scientific were prepared. Electrodes of a multi-point electrode array (MEA; SCREEN Holdings Co., Ltd.) suspended in a medium and pre-coated with 0.05% polyethyleneimine (PE1; manufactured by Sigma) and 80 μg mL laminin solution (manufactured by Sigma) were used. After seeding on substrates, the cells were cultured for one week, and then lentivirus LV-Synapsin-GFP (manufactured by SignaGen Laboratories) was added to the medium to force expression of GFP.

It was confirmed that synchronous firing of neurons was induced by the formation of a network after 3 weeks of culture.

### Arrangement of Cells

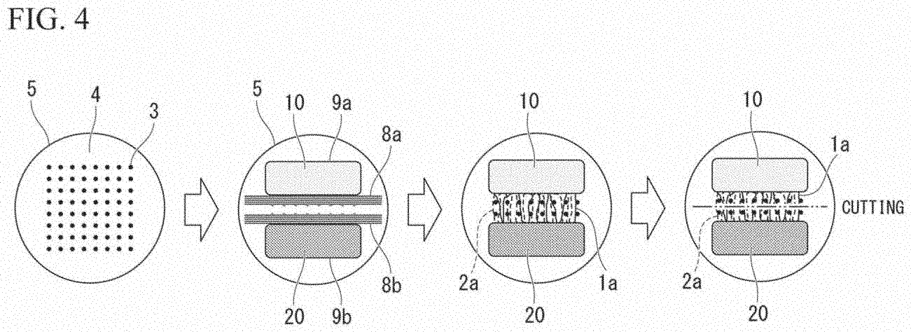

FIG. 4 is a figure of diagram showing the test flow in Example 1. First, a substrate 5 is prepared, attached partition members 8a and 8b made of dimethylpolysiloxane (PDMS) and frame members 9a and 9b were provided so as to separate a region containing cell population A (10) and a region containing cell population B (20). Next, on the substrate 5, two types of cell suspensions each containing a cell population A(10) and a cell population B (20) obtained by dividing the same neurons into two populations were applied. Seeds were sown in each region surrounded by the frame members 9a and 9b so as not to spread. Each cell suspension was seeded using a micropipette when the area of the region was larger than 10 mm². When the area was as narrow as 10 mm² or less, because it was difficult to seed by hand, each cell suspension was seeded using an inkjet bioprinter for cell ejection.

After confirming that the cells had adhered to the substrate 5, one day after the culture, the PDMS partition members 8a and 8b and the frame members 9a and 9b were peeled off to continue the culture. Cell bodies contained in cell population A (10) and cell population B (20) extended neurites while remaining in their respective regions, and a network in which the regions were connected to each other for a period of 1 week or more and 2 weeks or less was formed.

### Electrical Activity Measurement Step before Cutting

Cell population A and cell population B were cultured for 4 weeks in a state in which they were connected via neurites (a state in which a neural network was formed), and action potentials detectable from electrodes were measured using the MED64 system (SCREEN Holdings Co., Ltd.). Measurements were taken for 5 minutes. Analysis of electrical waveforms was performed with the software BurstScope attached to the MED64 system.

### Cutting Step

After the electrical activity measurement step, the neurites were cut with a scalpel (Feather Safety Razor Co., Ltd.) attached to a micromanipulator as shown in FIG. After cutting, the cells were incubated in a CO₂ incubator for 30 minutes to stabilize,

### Electrical Activity Measurement Step after Cutting

After 30 minutes from the cutting, the electrical activity measurement step described above was performed again.

### Observation Step

After the cutting step, the cut site was observed by time-lapse imaging. Specifically, using a plate reader Cytation5 (BioTek), continuous brightfield and fluorescence photography was performed for 3 days under an environment of 37° C. and 5% CO₂. In order to prevent the cells from being affected by drying during measurement, a BEMCOT containing sterilized water for humidification was placed around the sample, and to reduce phototoxicity, BrainPhys Imaging Optimized Medium (STEMCELL Technologies), a medium suitable for fluorescence observation, was used. A general GFP observation filter and LED were used for fluorescence observation with lentivirus, and a 10× objective lens was used.

### Measurement Result

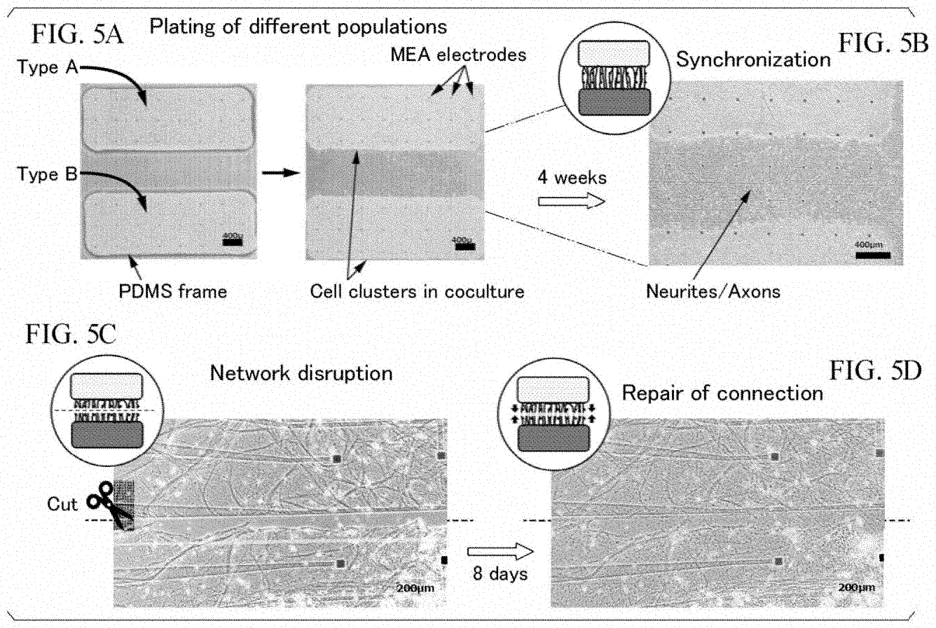

FIG. 5(a) shows micrographs when the cell populations A and B were arranged, and FIG. 5(b) shows micrographs after culturing for 4 weeks. FIG. 5(c) shows micrographs of neurites at the time of neurite cutting, and FIG. 5(d) shows micrographs of elongated and regenerated neurites eight days after the cutting step.

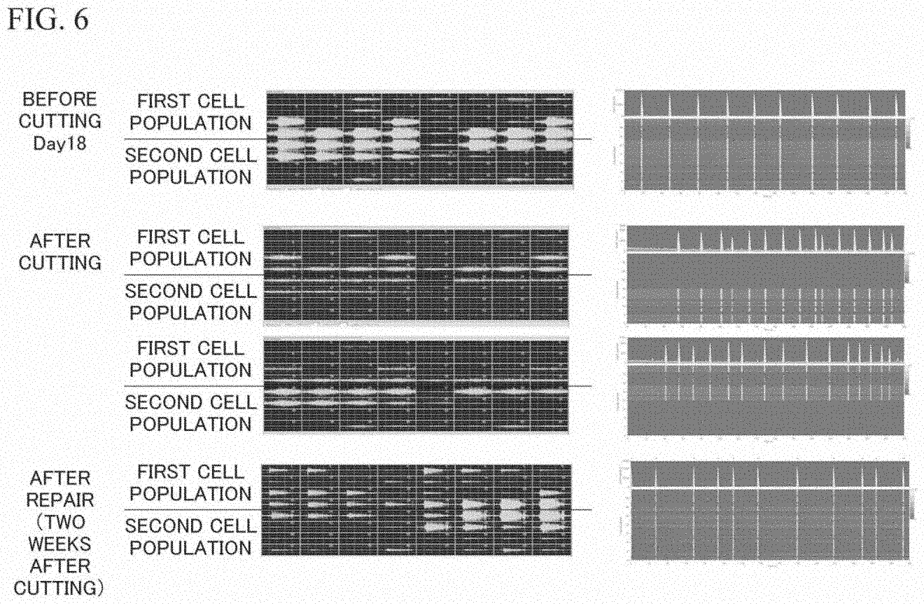

FIG. 6 shows the measurement data of electrical activity by the multi-point electrode array system (MED64 Alphamed Scientific Inc.) for the electrical activity measurement step and shows the result of measuring the electrical activity before and after the cutting step. From the right figure, it can be seen that the electrical activities of the neuronal populations were synchronized before neurite transection, but not after neurite transection. When the culture was continued and the electrical activity was continuously measured, it was observed that the synchronization of the electrical activity between the neuronal populations began to recover in about one week. Two weeks after cutting, synchronization of electrical activity between neuronal populations was restored.

### Example 2

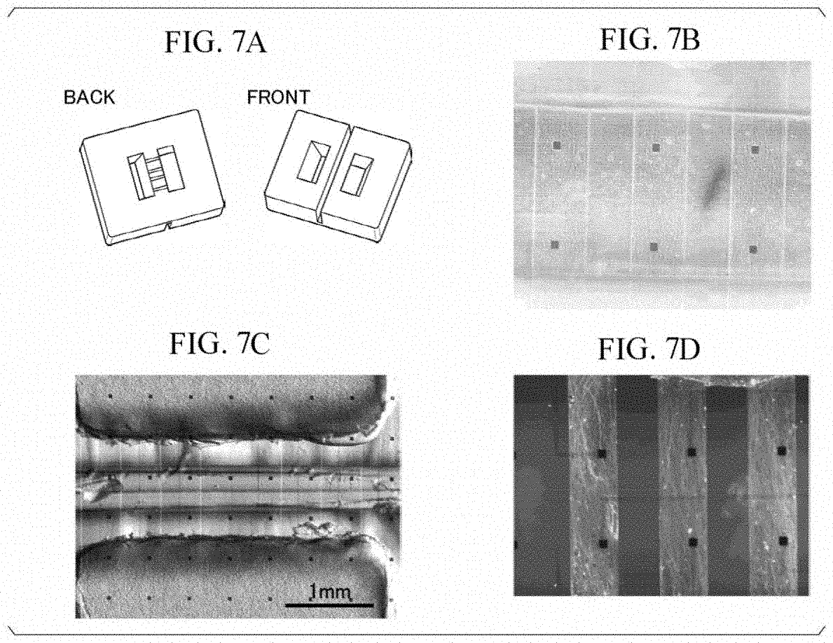

FIG. 7 shows a method for evaluating neuronal functional connections using another jig. PDMS-fabricated jigs can be used to control the spatial arrangement of cell bodies and neurites. As shown in FIG. 7(a), the jig of this example has microtunnels (μm-order height) in the PDMS jig, and cell bodies cannot penetrate, but cell projections can penetrate; therefore, the spatial location of cell bodies and neurites can he controlled. In the neurite-severed cell population, neurons may migrate and reestablish functional connections, but this jig makes it possible to ignore the contribution of neuronal migration to repair.

FIG. 7(b) shows a 10×micrograph of cell culture (arrangement of cell populations) using the jig in FIG. 7(a), and FIG. 7(c) shows a 2× micrograph. FIG. 7(d) shows the results of image observation performed in the same manner as in Example 1. This example showed that re-extension of neurites could be observed in more detail.

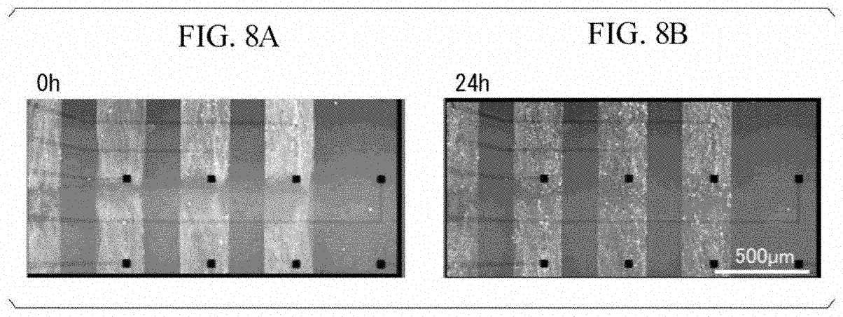

FIG. 8 shows a photograph of image observation in the observation step using the jig in FIG. 7. FIG. 8(a) shows from immediately after the cutting step to several hours after, and FIG. 8(b) shows 24 hours after the cutting step. Although the neurites were cut with a scalpel, it can be seen that the spatial gaps between the neurites were closed in about 24 hours.

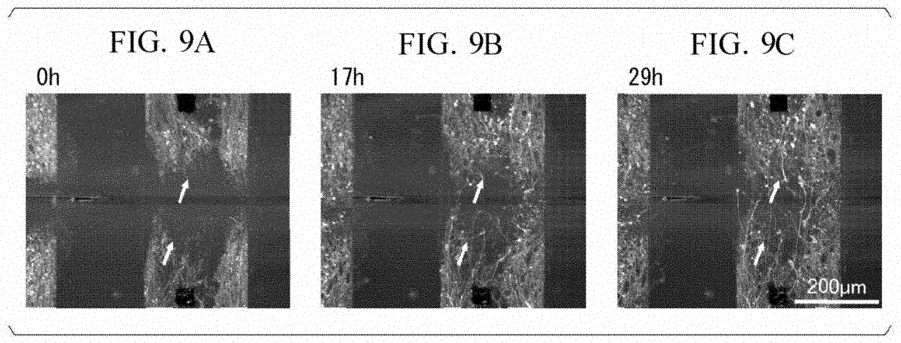

FIG. 9 shows the results of similar image observations (time-lapse imaging) performed over time. FIG. 9(a) shows the observation results immediately after the cutting step, FIG. 9(b) shows the observation results after 17 hours, and FIG. 9(c) shows the observation results after 29 hours. It was observed that the neurite was extending again from the projection. By observing the process extension of individual neurons, it is possible to determine what kinds of neurons contribute to repair.

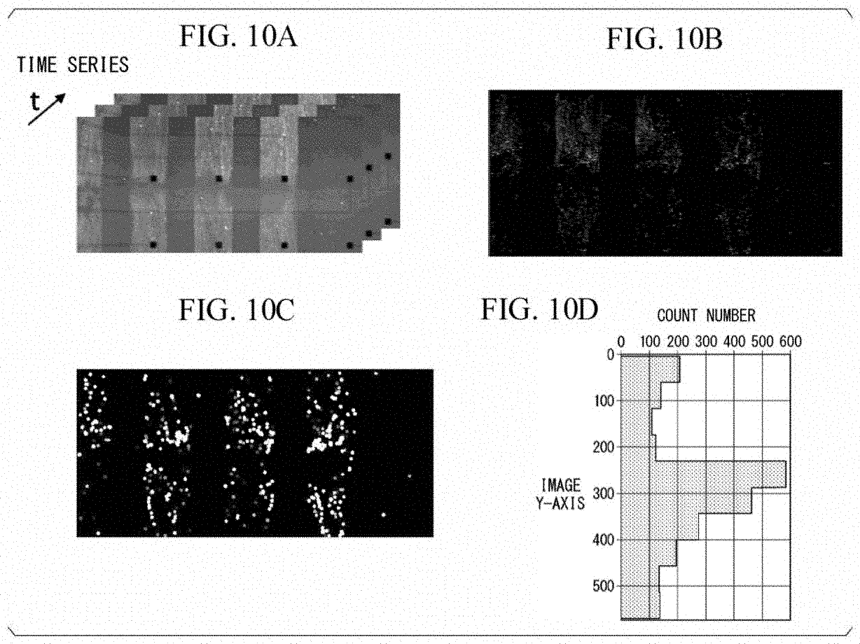

FIG. 10 shows the results of further detailed analysis of the images obtained by time-lapse imaging. In time-lapse imaging, it is possible to observe changes over time by taking pictures of an object at different times and arranging them in time series (FIG. 10(a)). By detecting the difference between two images (FIGS. 10(b) and 10(c)), it is possible to visualize the location that has moved over time. With the direction of the tunnel (vertical direction) of the image as the y-axis, the positions of the protrusions on the y-coordinate were counted during the observation period of 72 hours, and the summarized results are shown in FIG. 10(d). It can be seen that many neurites are moving in the vicinity of the amputation site with respect to the y-axis of the image.

From the above observation results, it was found that although the spatial gap itself at the cut point was repaired in about 24 hours, it took about another week for the electrical functional connection to recover. It can be seen that a more detailed analysis of the repair function is possible by analyzing the physical state of repair (for example, analysis of the shape, dynamics, elongation speed, etc. of protrusions) and electrical functional measurement analysis.

The present invention includes the following aspects.

[1] A method for evaluating neuronal functional connectivity in vitro, comprising:

for multiple cell populations that are spaced apart from each other and connected via neurites,

a cutting step of cutting the neurites;

an observation step of observing the dynamics of neurons whose neurites have been cut; and

an electrical activity measurement step of measuring the electrical activity of each of the plurality of cell populations before and after the cutting step,

wherein at least one of the cell populations contains neurons.

[2] The method for evaluating neuronal functional connectivity according to [1], wherein the observation step uses a camera to observe the dynamics of neurons.
[3] The method for evaluating neuronal functional connectivity according to [1] or [2], wherein all of the plurality of cell populations contain neurons.
[4] The method for evaluating neuronal functional connections according to any one of [1] to [3], wherein the neurons are differentiation-induced from stem cells.
[5] The method for evaluating neuronal functional connectivity according to any one of [1] to [4], wherein the plurality of cell populations are all derived from stem cells.
[6] The method for evaluating neuronal cell functional connectivity according to any one of [1] to [5], wherein the stem cells are induced pluripotent stem cells.
[7] The method for evaluating neuronal functional connectivity according to any one of [1] to [6], wherein the electrical activity measurement step performs at least one measurement each before and after the cutting step.

### EXPLANATION OF REFERENCES

1: Neurons

1a, 2a, 2b, 2c: Neurites

2: Cells that transmit electrical signals to neurons

3, 3a, 3b, 3c, 3d, 3e, 3f: detectors (electrodes)

4: medium

5: Substrate (culture vessel)

6a, 6b, 6c, 7a, 7b, 7c: Wiring

8a, 8b: Partition member

9a, 9b: Frame members

10: First cell population (cell population A)

20: Second cell population (cell population B)

30: Third cell population

40: Fourth cell population

100: Cell substrate

### PRIOR ART DOCUMENTS

### Patent Document

[Patent Document 1] Japanese Patent Publication No. 2020-156463A

### Non-Patent Literature

[Non-Patent Document 1] van de Wijdeven et al., A novel lab-on-chip platform enabling axotomy and neuromodulation in a multi-nodal network, Biosensors and Bioelectronics 140 (2019) 111329

1. A method for evaluating neuronal functional connectivity in vitro, for a plurality of cell populations that are spaced apart from each other and connected via neurites, the method comprising:
cutting the neurites;
observing the dynamics of neurons whose neurites have been cut; and
measuring the electrical activity of each of the plurality of cell populations before and after the cutting,
wherein at least one of the cell populations contains neurons.

2. The method for evaluating neuronal functional connectivity according to claim 1, wherein the observing uses a camera to observe the dynamics of neurons.

3. The method for evaluating neuronal functional connectivity according to claim 1, wherein all of the plurality of cell populations contain neurons.

4. The method for evaluating neuronal functional connectivity according to claim 1, wherein the neurons are induced to differentiate from stem cells.

5. The method for evaluating neuronal functional connectivity according to claim 1, wherein the plurality of cell populations are all derived from stem cells.

6. The method for evaluating neuronal functional connectivity according to claim 1, wherein the stem cells are induced pluripotent stem cells.

7. The method for evaluating neuronal functional connectivity according to claim 1, wherein the measuring comprises at least one measurement each before and after the cutting.
